(19) United States
(12) Patent Application Publication
(10) Pub. No.: US 2019/0048302 A1
(43) Pub. Date: Feb. 14, 2019

(54) CELL CULTURE VESSEL AND JIG FOR FIXING CELL CULTURE VESSEL

(71) Applicant: FUKOKU CO., LTD., Saitama (JP)

(72) Inventors: Ikumi Suzuki, Saitama (JP); Takashi Morimura, Saitama (JP); Takao Yoshida, Saitama (JP)

(73) Assignee: FUKOKU CO., LTD., Saitama (JP)

(21) Appl. No.: 16/079,686

(22) PCT Filed: Feb. 14, 2017
(86) PCT No.: PCT/JP2017/005334

§ 371 Date: Aug. 24, 2018

(30) Foreign Application Priority Data
Feb. 25, 2016 (JP) 2016-034819

(51) Int. Cl. C12M 1/00; C12M 3/00; C12M 1/04

(52) U.S. Cl. CPC C12M 1/007; C12M 23/14; C12M 1/08; C12M 23/24; C12M 29/04; C12M 23/48

(57) ABSTRACT

The present invention provides a cell culture vessel and a jig for fixing the cell culture vessel in position. More specifically, the cell culture vessel of the present invention is provided with a vessel portion having an annular sealed culture space when viewed from overhead and a port portion connecting the inner portion and outer portion of the culture space. The vessel portion is provided with a first vessel wall serving as the upper side during culturing and a second vessel wall serving as the lower side during culturing. Preferably at least the first vessel wall has flexibility.

### TECHNICAL FIELD

The present invention relates to a cell culture vessel provided with a sealed culture space and to a jig for fixing the cell culture vessel in position.

### BACKGROUND ART

Research and development on ES cells and iPS cells in particular has advanced in recent years. These advances have made it possible to produce various cells resulting in high expectations being placed on cell therapy and regenerative therapy using these cells. In addition, there is also a growing demand for new cell culture technologies for stably and easily enabling cells used in these therapies to be acquired in large numbers.

For example, the cultured cells may be used for medical purposes. In that case, the culture vessels used preferably have the properties indicated below:
high level of culture performance;
allows culture status to be easily observed;
cultured cells resistant to bacterial contamination; and
ease of handling.

Namely, it is preferable to use a culture vessel that demonstrates superior aseptic manipulation.

From the viewpoint of aseptic manipulation, pouch-like vessels (bags) have been attempted to be used as vessels for cell culturing instead of conventional Petri dishes and flasks. These pouch-like vessels (bags) are provided with a port for filling and discharging the content liquid and a tube that connects to the port. The pouch-like vessel has, for example, vessel walls having high gas permeability. Cells are cultured within the pouch-like vessel. Oxygen required for cell growth is supplied through the vessel walls. One of the metabolites of cells in the form of carbon dioxide is discharged through the vessel walls.

This type of pouch-like vessel is described in JP 2009-027944A.

In addition, a cell culture vessel preferable for shake culturing is described in JP 2010-136628A. This vessel has an oblong shape when viewed from overhead. This vessel is placed on a shaking table (seesaw). Culture broth inside the vessel teeters back and forth due to rocking of the shaking table.

### PRIOR ART DOCUMENTS

### Patent Documents

### Patent Document 1: JP 2009-027944A

### Patent Document 2: JP 2010-136628A

### DISCLOSURE OF THE INVENTION

### Problems to be Solved by the Invention

Conventional cell culture vessels are provided with a sealed culture space. However, conventional cell culture vessels are not suitable for rotary culturing. Rotary culturing refers to a method for generating a rotational flow in a liquid culture medium in a vessel by rotating the culture vessel horizontally. In addition, there are also cases in which, depending on the purpose of the cell culturing, it is desirable to inhibit the generation of eddying flow in the liquid medium towards the center of the culture vessel during rotation of the culture vessel.

With the foregoing in view, an object of the present invention is to provide a cell culture vessel preferable for rotary culturing. An object of the present invention is to provide a cell culture vessel capable of inhibiting generation of eddying flow in the liquid medium towards the center of the culture vessel during rotation of the culture vessel. In addition, an object of the present invention is to provide a fixing jig that clamps the cell culture vessel in position.

### Means for Solving the Problems

The present invention is as indicated below.

[1] A cell culture vessel, comprising:

a vessel portion having an annular sealed culture space when viewed from overhead; and

a port portion connecting the inner portion and outer portion of the culture space; wherein,

the vessel portion is provided with a first vessel wall serving as the upper side during culturing and a second vessel wall serving as the lower side during culturing.

[2] The cell culture vessel described in [1], wherein the first vessel wall has flexibility.

[3] The cell culture vessel described in [1], wherein both the first vessel wall and the second vessel wall have flexibility. [4] The cell culture vessel described in [1], wherein the first vessel wall has flexibility and the second vessel wall has shape-retaining property.

[5] The cell culture vessel described in [4], provided with an annular outer peripheral wall rising from the outer periphery of the second vessel wall and an annular inner peripheral wall rising from the inner periphery of the second vessel wall.

[6] The cell culture vessel described in [1], wherein both the first vessel wall and the second vessel wall have shape-retaining property.

[7] The cell culture vessel described in any of [2] to [5], wherein the inner surface of the first vessel wall and the inner surface of the second vessel wall are substantially adhered to each other when contents containing a gas are discharged from inside the vessel portion.

[8] The cell culture vessel described in any of [1] to [7], wherein at least one of the first vessel wall and the second vessel wall has oxygen permeability.

[9] A jig for fixing the cell culture vessel described in any of [1] to [8] in position, comprising:

a first plate and a second plate for clamping the vessel portion, and

holding means for holding the first plate and the second plate in a state of clamping the vessel portion.

### Effects of the Invention

Since the cell culture vessel of the present invention is provided with a vessel portion having a sealed culture space, the risk of contamination during cell culturing can be reduced.

The cell culture vessel of the present invention is able to allow the generation of a rotary flow along an annular culture space in a liquid medium within the culture vessel during rotation of the vessel. Consequently, the cell culture vessel of the present invention is preferable for rotary culturing.

Moreover, the cell culture vessel of the present invention has an inner peripheral edge or inner peripheral wall that forms an annular culture space. Consequently, according to the present invention, the generation of eddying flow in the liquid medium towards the center of the vessel portion can be inhibited.

### BRIEF DESCRIPTION OF THE DRAWINGS

FIG. 1 is a perspective view showing one embodiment (inner tube-shaped bag) of the cell culture vessel according to the present invention.

FIG. 2 is a perspective view showing another embodiment (double circular tray-shaped bag) of the cell culture vessel according to the present invention.

FIG. 3 is a schematic cross-sectional view of the vessel portion of the cell culture vessel (double circular tray-shaped bag) shown in FIG. 2.

FIG. 4 is a perspective view showing the state of the cell culture vessel shown in FIG. 1 (inner tube-shaped bag) fixed in position with a jig.

FIG. 5 is a side view showing the state of the cell culture vessel shown in FIG. 1 (inner tube-shaped bag) fixed in position with a jig.

FIG. 6 is an exploded view showing the state of the cell culture vessel shown in FIG. 1 (inner tube-shaped bag) fixed in position with a jig.

### MODE FOR CARRYING OUT THE INVENTION

The cell culture vessel according to an embodiment of the present invention is provided with a vessel portion having an annular sealed culture space when viewed from overhead and a port portion connecting the inner portion and outer portion of the culture space. The vessel portion is provided with a first vessel wall serving as the upper side during culturing and a second vessel wall serving as the lower side during culturing. Among the first vessel wall and the second vessel wall, at least the first vessel wall preferably has flexibility. The first vessel wall is formed, for example, with a soft sheet.

Furthermore, in the case the cell culture vessel according to an embodiment of the present invention is used for rotary culturing, the sealed culture space having an annular shape when viewed from overhead is an annular flow path. In addition, in the case the cell culture vessel is similarly used for rotary culturing, the inner diameter of the culture space is determined, for example, to be within a range capable of inhibiting the generation of eddying flow in a liquid medium towards the center of the culture vessel during rotation.

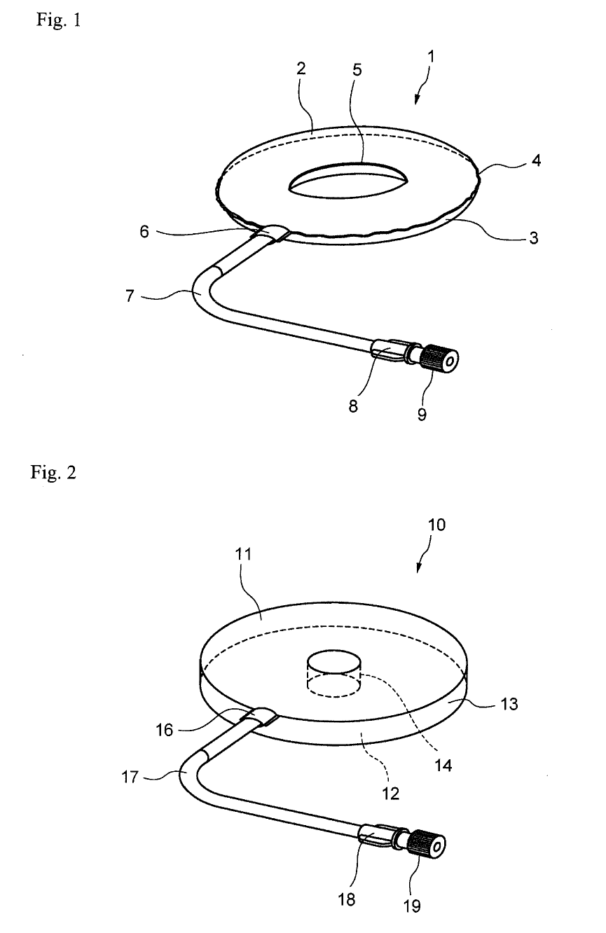

In an embodiment of the present invention, both the first vessel wall and the second vessel wall may have flexibility. For example, the first vessel wall and the second vessel wall may be formed with a soft sheet. A inner tube-shaped bag shown in FIG. 1 is one example of a cell culture vessel having such a first vessel wall and a second vessel wall.

In another embodiment of the present invention, the first vessel wall may have flexibility. For example, the first vessel wall may be formed with a soft sheet. The second vessel wall may have shape-retaining property. For example, the second vessel wall may be formed with a hard sheet.

The second vessel wall may be provided with an annular outer peripheral wall rising from the outer periphery thereof and an annular inner peripheral wall rising from the inner periphery thereof. The second vessel wall may be formed with a hard sheet.

The double circular tray-shaped bag shown in FIG. 2 is an example of a cell culture vessel having such a second vessel wall. Furthermore, the second vessel wall of the double circular tray-shaped bag may also be formed with a soft sheet provided it has shape-retaining property.

In another embodiment of the present invention, both the first vessel wall and the second vessel wall may have shape-retaining property. For example, the first vessel wall and the second vessel wall may be formed with a hard sheet.

The thicknesses of the first vessel wall and the second vessel wall may be determined, for example, according to the size of the inner diameter and outer diameter of the cell culture vessel or the weight of the liquid medium supplied to the vessel portion. The thicknesses of the first vessel wall and the second vessel wall are not limited to specific ranges by the term “sheet”.

In the inner tube-shaped bag shown in FIG. 1, the thicknesses of the first vessel wall and the second vessel wall may be the same or different. The thicknesses of the first vessel wall and the second vessel wall may be determined, for example, according to the inner diameter and outer diameter of the bag or the weight of the liquid medium supplied to the bag. The determined thickness of the second vessel wall may be greater than the thickness of the first vessel wall. This applies similarly to the double circular tray-shaped bag shown in FIG. 2.

The first vessel wall and the second vessel wall may also have flexibility. Here, “flexibility” refers to, for example, conformability of a vessel wall. In the case a vessel wall has conformability, the liquid medium or other content liquid is able to flow into or flow out from the inside of the vessel portion in the absence of the flow of air into or from the vessel portion. This is to secure the volume of the vessel portion by allowing the vessel wall having conformability to deform. Conformability of a vessel wall is not merely determined by a physical value of the material used to form the vessel walls. Conformability of a vessel wall changes according to such factors as the shape of the vessel and the amount of air present within the vessel. Thus, it is generally not appropriate to limit the present invention by a physical value of the material used to form the vessel walls.

At least the first vessel wall may have flexibility.

The first vessel wall having flexibility deformed and the internal volume of the vessel portion increases as a result of supplying liquid medium or air and the like to the vessel portion via the port portion. On the other hand, the first vessel wall having flexibility deforms and the inner volume of the vessel portion decreases as a result of discharging liquid medium or air and the like from the vessel portion via the port portion.

The inner surface of the first vessel wall and the inner surface of the second vessel wall are preferably substantially adhered to each other when contents such as liquid medium or air are discharged from inside the vessel portion.

In the inner tube-shaped bag shown in FIG. 1, the vessel portion expands and takes on a three-dimensional shape as a result of supplying a liquid medium or air and the like to the vessel portion. The vessel portion shrinks and takes on a flat shape as a result of discharging a liquid medium or air and the like from the vessel portion. Consequently, the change in internal volume of the vessel portion is large due to the supply and discharge of liquid medium or air and the like.

In the double circular tray-shaped bag shown in FIG. 2, as will be described later, a three-dimensional vessel portion is formed initially. Consequently, the change in internal volume of the vessel portion resulting from the supply and discharge of liquid medium or air and the like is typically smaller in comparison with the inner tube-shaped bag.

In the inner tube-shaped bag shown in FIG. 1, a gas such as air can be further supplied to the vessel portion after having supplied a content liquid such as liquid medium to the vessel portion. Since the inner surface of the first vessel wall easily separates from the liquid surface of the liquid medium as a result of supplying the gas, rotational flow of the liquid medium and unnecessary interference with the inner surface of the first vessel wall can be prevented.

Similarly, in the double circular tray-shaped bag shown in FIG. 2, a gas such as air can be further supplied to the vessel portion after having supplied a content liquid such as a liquid medium to the vessel portion. Since the inner surface of the first vessel wall easily separates from the liquid surface of the liquid medium as a result of supplying the gas, rotation flow of the liquid medium and unnecessary interference with the inner surface of the first vessel wall can be prevented.

On the other hand, the second vessel wall may have shape-retaining property. Here, “shape-retaining property” refers to the absence of a substantial change in the shape of the second vessel wall even if a content liquid such as a liquid medium, for example, is supplied to the vessel portion.

The content liquid flows during rotation of the vessel portion. The second vessel wall is able to be deformed by this flow of the content liquid.

In addition, the cell culture vessel is lifted up during, for example, cell observation or media replacement. When the vessel is lifted up, the second vessel wall is able to be deformed by the weight of the content liquid.

This deformation can be suppressed as a result of the second vessel wall having shape-retaining property.

Furthermore, the weight of the liquid medium inside the vessel portion increases as the diameter of the cell culture vessel (vessel portion) becomes larger. Consequently, even in the case of the second vessel wall having shape-retaining property, the second vessel wall may be bent to a certain degree by the weight of the content liquid when the cell culture vessel (vessel portion) is lifted up.

In the case the second vessel wall has shape-retaining property, the portion of the second vessel wall that serves as the bottom of the annular flow path is preferably flat in the manner of the bottom of a conventional culture dish.

The material that composes the first vessel wall and the second vessel wall of the cell culture vessel may be any arbitrary material. The material is a natural resin or synthetic resin, and preferably a synthetic resin, from the viewpoints of such factors as formability, economy and handling.

Examples of synthetic resins include polystyrene resin, polyester resin, polycarbonate resin, polymethyl methacrylate resin, cyclic olefin resin, polyethylene resin, ethylene-vinyl acetate copolymer resin, polypropylene resin and mixtures thereof. For example, the second vessel wall may be formed with a single layer sheet composed of these resins or a composite sheet containing these resins. Here, a composite sheet refers to a sheet composed of a plurality of resins. Examples of sheet composed of a plurality of resins include sheets composed of a resin mixture, laminated films, resin films coated with resin and resin films having a resin printed thereon.

The first vessel wall and the second vessel wall of the cell culture vessel may be composed with a soft sheet. Examples of resins used to form a soft sheet include low-density polyethylene resin, ethylene-vinyl acetate copolymer resin, polypropylene resin, ethylene-propylene copolymer resin, polybutadiene resin, styrene-butadiene copolymer resin and hydrogenated resins thereof, polyurethane resin, and mixtures thereof. These resins may also be used to form a hard sheet.

Low-density polyethylene naturally includes not only ordinary low-density polyethylene, but also linear low-density polyethylene resin and metallocene-catalyzed low-density polyethylene resin. Polypropylene resin includes stereoblock polypropylene resin and mixtures of polypropylene resin and stereoblock polypropylene resin.

The surfaces of the insides (sides facing the culture space) of the first vessel wall and second vessel wall are preferably hydrophobic surfaces in order to prevent adhesion of cells. The surfaces of the insides (sides facing the culture space) of the first vessel wall and second vessel wall are preferably formed from a hydrophobic resin. Alternatively, the surfaces of the insides (sides facing the culture space) of the first vessel wall and second vessel wall are preferably preliminarily subjected to hydrophobic treatment or treatment that inhibits cell adhesion. Examples of such treatment include agarose coating, poly(hydroxyethyl methacrylate) (poly-HEMA) coating and 2-methacryloyloxyethyl phosphorylcholine (MPC) coating.

The surfaces of the insides (sides facing the culture space) of the first vessel wall and second vessel wall are not limited to hydrophobic surfaces. The surfaces of the insides (sides facing the culture space) of the first vessel wall and second vessel wall may be hydrophobic surfaces or hydrophilic surfaces. Whether or not the surfaces are hydrophobic or hydrophilic may be determined by the type of cells cultured, the type of liquid medium, additives, size of the culture vessel and rotating speed of the culture vessel.

In the case hydrophilic surfaces are preferable, the surfaces on the insides of the first vessel wall and second vessel wall are formed from a hydrophilic resin. Alternatively, the surfaces of the insides of the first vessel wall and second vessel wall are preliminarily subjected to hydrophilic treatment or treatment that promotes cell adhesion. Examples of such treatment include corona discharge treatment that adds hydroxyl groups and carboxyl groups to a surface, collagen coating and poly-D-lysine coating.

In addition, at least one of the first vessel wall and second vessel wall of the culture vessel preferably has gas permeability. In particular, at least one of the first vessel wall and second vessel wall of the culture vessel is preferably permeable to oxygen and carbon dioxide. The supply of oxygen required by cells to the vessel and the discharge of carbon dioxide outside the vessel can be carried out through the vessel walls. Since the inside and outside of the vessel are not in direct communication, sterility can be maintained within the vessel.

Here, gas permeability refers to mainly the permeability of oxygen and carbon dioxide. Oxygen permeability and carbon dioxide permeability tend to be similar for each type of material. Moreover, carbon dioxide permeability is much greater in comparison with oxygen permeability. For such reasons, the gas permeability of a vessel used to culture cells is typically evaluated according to oxygen permeability.

Thus, the first vessel wall and the second vessel wall may have oxygen permeability. Oxygen permeability changes according to the number of cultured cells and the area of the vessel walls through which oxygen passes. Normally, the oxygen permeability required of the vessel walls is proportional to the number of cells cultured and inversely proportional to the area of the vessel walls through which oxygen passes.

The first vessel wall and the second vessel wall may each have different oxygen permeability. For example, in the case of having cultured cells with the highest efficiency, the number of cultured cells can be assumed to be proportional to the amount of medium. A value is obtained by dividing the sum of the oxygen permeability per effective area of the first vessel wall at 25° C. and the oxygen permeability per effective area of the second wall at 25° C. by the amount of content liquid. A culture vessel can be designed so that the value is equal to or greater than 0.2 cc/atm·day·ml. The designed vessel is able to prevent a shortage of oxygen supplied to cells during cell culturing.

Here, oxygen permeability is as indicated below.

A value representing oxygen permeability is obtained by dividing the amount of oxygen that passes through a vessel wall by the area of the vessel wall when continuously applying a differential oxygen pressure of 1 atm to the vessel wall for 24 hours at 25° C.

Oxygen permeability per effective area of a vessel wall is a value obtained by multiplying the area of the vessel wall by the oxygen permeability of the vessel wall.

Oxygen permeability per effective area of a vessel wall represents the ability of the vessel wall to supply oxygen to the inside of the vessel.

The total oxygen permeability per effective area of all vessel walls contained in a single vessel represents the ability of that vessel to supply oxygen to the inside of the vessel.

Moreover, a value representing the amount of oxygen able to be supplied by the cell culture vessel of the present invention per 1 ml of medium is obtained by dividing the total oxygen permeability per effective area by the amount of liquid medium supplied to the vessel.

The following provides a detailed explanation of embodiments of the present invention based on the drawings.

FIG. 1 is a perspective view showing a cell culture vessel (inner tube-shaped bag) 1 according to one embodiment of the present invention. Furthermore, FIG. 1 indicates the state in which a liquid medium seeded with cells and a prescribed amount of air that has passed through a sterile filter are injected into the cell culture vessel 1 thereby causing the cell culture vessel 1 to expand.

The vessel portion of the cell culture vessel 1 is provided with a first vessel wall 2 having an annular shape when viewed from overhead and a second vessel wall 3 having roughly the same shape. The first vessel wall 2 and the second vessel wall 3 are composed by flat soft sheets having flexibility. The flat soft sheets composing the first vessel wall 2 and the second vessel wall 3 are mutually joined at their respective outer peripheral edges and inner peripheral edges. As a result, the vessel portion composes a sealed culture space in the form of a flat ring (inner tube shape) having an outer peripheral edge 4 and an inner peripheral edge 5. Furthermore, both the first vessel wall 2 and the second vessel wall 3 are formed with a soft sheet.

The vessel portion of the cell culture vessel 1 has a flat shape as a result of the inner surface of the first vessel wall 2 and the inner surface of the second vessel wall 3 being substantially adhered to each other prior to the supply and after the discharge of contents containing a gas.

When contents containing a gas are supplied to the vessel portion of the cell culture vessel 1, the vessel portion expands and takes on the three-dimensional shape of a inner tube due to the flexibility of the first vessel wall 2 and the second vessel wall 3.

The first vessel wall 2 and the second vessel wall 3 may be composed with a soft sheet. The soft sheet has flexibility and oxygen permeability, and an example thereof is a single layer sheet having a thickness of 140 μm composed of an ethylene-vinyl acetate copolymer resin. Furthermore, composite sheets thereof may also be used for the second vessel wall of a cell culture vessel (double circular tray-shaped bag) to be subsequently described.

For example, the diameter of the outer periphery of the cell culture vessel 1 is 80 mm and the diameter of the inner periphery is 45 mm.

In the case the first vessel wall 2 and the second vessel wall 3 have flexibility, the inner surface of the first vessel wall 2 and the inner surface of the second vessel wall 3 can be easily adhered to each other. As a result of allowing the inner surface of the first vessel wall 2 and the inner surface of the second vessel wall 3 to adhere to each other, liquid medium can be easily discharged from the vessel portion when replacing the liquid medium, for example.

The annular vessel portion has a port to be subsequently described. A portion of the vessel portion on the side opposing the port can be clamped with a clip and the like. A portion of the first vessel wall 2 and a portion of the second vessel wall 3 on the side opposing thereto are temporarily able to be in contact with each other by clamping the vessel portion with a clip and the like. Clamping a portion of the vessel portion with a clip and the like temporarily divides the space within the annual vessel portion into a large space on the port side and a small space on the side not having the port. Cells can be temporarily isolated during or after culturing in the small space on the side not having the port. Isolating cells during or after culturing enables only liquid medium to be easily discharged or replaced.

The port portion of the cell culture vessel 1 is at least provided with a port 6 that connects the inside and outside of the culture space of the vessel portion. Moreover, the port section may also be provided with a tube 7 connected to the open end of the port 6, an outlet port 8 provided on the end of the tube 7, and a cap 9 attached to the end of the outlet port 8.

Furthermore, in the case the port portion is only composed of the port 6, a cap is preferably attached to the end of the port 6. The end of the port 6 may be closed off by heat sealing instead of attaching a cap.

A liquid medium seeded with cells and a prescribed amount of air that has passed through a sterile filter can be injected into the vessel portion of the cell culture vessel 1. The vessel portion expands when these components are injected into the vessel portion. When the vessel portion expands, the vessel portion takes on the three-dimensional shape of an inner tube. When the vessel portion is placed on the table surface of a rotating device, the inner tube-shaped vessel portion contacts the table surface over a circular region having a prescribed diameter.

Consequently, the vessel portion does not swing on the rotary table during rotation of the vessel portion. In addition, the vessel portion can be maintained in a stable position during rotation of the vessel portion.

When rotating the cell culture vessel 1, the cell culture vessel 1 may be set, for example, in the jig shown in FIG. 4. The width of the bottom of the vessel portion (annular flow path) can be increased in the radial direction by placing the cell culture vessel 1 in the jig and compressing the vessel portion. In addition, by setting the cell culture vessel 1 in the jig, deformation of the cell culture vessel 1 when lifted up can be prevented, which is favorable when, for example, observing cells. There are no particular limitations on this jig provided it does not have an effect on suspension culturing of the cells. For example, the jig may be equipped on a culturing apparatus or may be independent of a culturing apparatus.

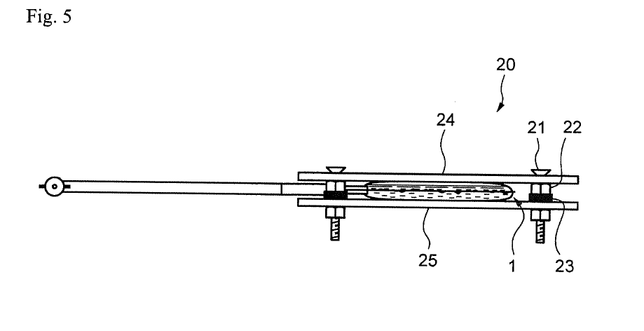

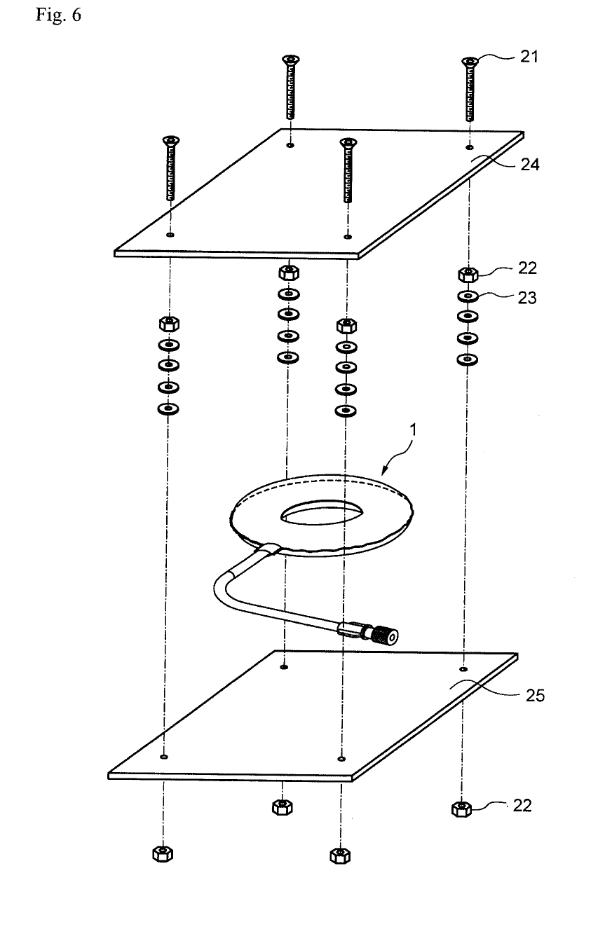

An example of fixing the cell culture vessel 1 in position using a jig that is independent from a culturing apparatus is shown in FIGS. 4 to 6. FIG. 4 is an overhead view showing the state in which the culture vessel 1 is fixed in position with a jig 20. FIG. 5 is a side view thereof. FIG. 6 is an exploded view thereof

The jig 20 is provided with a first plate 24, a second plate 25, connecting bolts 21, nuts 22 and washers 23. The first plate 24 and the second plate 25 clamp the vessel portion of the cell culture vessel 1. The first plate 24 and the second plate 25 are connected by the connecting bolts 21 and nuts 22 via bolt holes provided in the four corners of each plate. The gap between the first plate 24 and the second plate 25 is adjusted with the washers 23.

The gap between the first plate 24 and the second plate 25 can be adjusted according to the number of washers 23 used. The first plate 24 and the second plate 25 are preferably transparent in order to observe the inside of the cell culture vessel 1.

Furthermore, the jig 20 shown in FIG. 5 may be inverted vertically. Namely, the jig 20 may be placed on the table surface so that the heads of the bolts 21 contact the table surface of the rotating device.

In addition, there are no limitations on the holding means for holding the first plate 24 and the second plate 25 in a state in which they clamp the vessel portion of the cell culture vessel 1. For example, the holding means are not limited to the connecting bolts 21, nuts 22 and washers 23. The holding means may employ any arbitrary configuration. For example, a tubular member (pipe) of a prescribed length may be used instead of the plurality of washers 23.

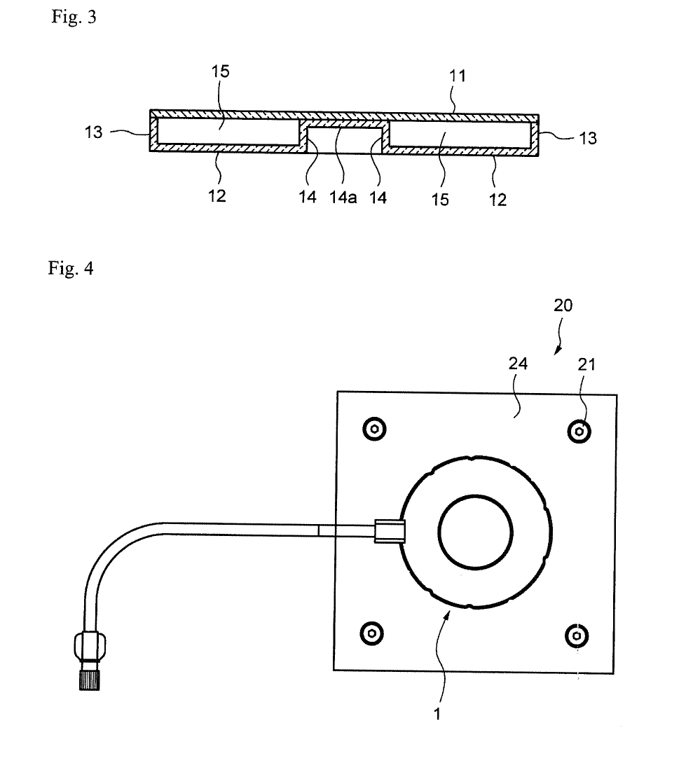

FIG. 2 is a perspective view of a cell culture vessel (double circular tray-shaped bag) 10 according to another embodiment of the present invention. FIG. 3 is a schematic cross-sectional view of the vessel portion of the cell culture vessel 10.

The vessel portion of the cell culture vessel 10 is composed of a first vessel wall 11 and a second vessel wall 12. The first vessel wall 11 is formed with a soft sheet and has flexibility. The second vessel wall 12 is formed with a hard sheet and has shape-retaining property.

The second vessel wall 12 is provided with an annular outer peripheral wall 13 that rises extending from the outer periphery and an annular inner peripheral wall 14 that rises extending from the inner periphery. The inner diameter side of the top portion of the inner peripheral wall 14 is closed at an upper surface 14a. In this manner, the outer peripheral wall 13 and the inner peripheral wall 14 are integrally formed with the second vessel wall 12.

In the present embodiment, the second vessel wall 12 is initially provided with the outer peripheral wall 13 and the inner peripheral wall 14 and is formed into the shape of a tray. In this manner, the second vessel wall 12 initially has a concave three-dimensional structure. As a result of the second vessel wall 12 having a three-dimensional structure, contents such as liquid medium can be supplied to the vessel portion without deforming the second vessel wall 12. Furthermore, the second vessel wall 12 may also be formed with a soft sheet provided it has shape-retaining property.

The heights of the outer peripheral wall 13 and the inner peripheral wall 14 are preferably about equal. In the present embodiment as well, the heights of the outer peripheral wall 13 and the inner peripheral wall 14 are about equal as shown in FIGS. 2 and 3. The top portion of the outer peripheral wall 13 of the second vessel wall 12 joins to the outer peripheral edge of the first vessel wall 11. The top portion of the inner peripheral wall 14 of the second vessel wall 12 may either be joined to the inner surface of the first vessel wall 11 or removably adhered thereto. Alternatively, the upper surface 14a on the inner diameter side of the top portion of the inner peripheral wall 14 may either be joined to the opposing inner surface of the first vessel wall 11 or may be removably adhered thereto. As a result thereof, an annular, sealed culture space 15 is composed when viewed from overhead.

The upper surface 14a on the inner diameter side of the top portion of the inner peripheral wall 14 need not be present provided the annular, sealed culture space 15 is composed when viewed from overhead. In this case, the top portion of the inner peripheral wall 14 is joined to the inner surface of the first vessel wall 11. In addition, in the case the top portion of the inner peripheral wall 14 is joined to the inner surface of the first vessel wall 11, a vessel wall is not required to be present closer to the inner diameter side than the joined location in the first vessel wall 11.

The second vessel wall 12 of the cell culture vessel 10 has shape-retaining property. The inner peripheral wall 14 and the upper surface 14a are formed in the second vessel wall 12. As a result of employing such a configuration, the annular, sealed culture space 15 is composed when viewed from overhead provided that at least the top portion of the outer peripheral wall 13 of the second vessel wall 12 joins to the outer peripheral edge of the first vessel wall 11.

Furthermore, instead of integrally providing the inner peripheral wall 14 in the second vessel wall 12, the bottom surface of the second vessel wall 12 may be flat in the manner of a culture dish. An annular member corresponding to the inner peripheral wall 14 may then be subsequently attached in the center of that bottom surface.

The soft sheet used to compose the first vessel wall 11 of the cell culture vessel 10 may be loose in the radial direction. In this case, the inner surface of the first vessel wall 11 and the inner surface (bottom surface) of the second vessel wall 12 substantially adhere to each other when contents containing a gas are discharged from inside the vessel portion.

The soft sheet used to compose the first vessel wall 11 of the cell culture vessel 10 may be loose in the radial direction. In this case, the first vessel wall 11 is able to expand upwards when air that has passed through a sterile filter is injected into the vessel portion.

The port portion of the cell culture vessel 10 is at least provided with a port 16 that connects the inner portion and outer portion of the culture space of the vessel portion. Moreover, the port portion may also be provided with a tube 17 connected to the open end of the port 16, an outlet port 18 provided on the end of the tube 17, and a cap 19 attached to the end of the outlet port 18.

Furthermore, in the case the port portion is only composed of the port 16, a cap is preferably attached to the end of the port 16. The end of the port 16 may be closed off by heat sealing instead of attaching a cap.

At least one of the first vessel wall 2 (11) and the second vessel wall 3 (12) of the cell culture vessel 1 (10) is preferably transparent. More preferably, the first vessel wall 2 (11) and the second vessel wall 3 (12) are both transparent.

When culturing cells, the shape, color, and other characteristics of the cells are observed with a microscope. In addition, the next procedure is typically carried out after having observed cell growth and cell status with a microscope.

Thus, at least the vessel wall on the side where the microscope is positioned is preferably transparent to a degree that enables the cells to be observed with the microscope. Namely, among the first vessel wall 2 (11) and the second vessel wall 3 (12), at least the vessel wall on the side where the microscope is positioned preferably has light transmittance of 80% or more.

The cell culture vessel 1 shown in FIG. 1 may be of an arbitrary size provided it can be used for rotary culturing. The size of the cell culture vessel 1 ranges, for example, from roughly an outer diameter of 40 mm×inner diameter of 30 mm to roughly an outer diameter of 2000 mm×inner diameter of 400 mm.

The cell culture vessel 10 shown in FIG. 2 may also be of an arbitrary size provided it can be used for rotary culturing. The size of the cell culture vessel 10 ranges, for example, from roughly an outer diameter of 40 mm×inner diameter of 30 mm×height of 5 mm to roughly an outer diameter of 2000 mm×inner diameter of 400 mm×height of 1000 mm.

In the cell culture vessel 1 shown in FIG. 1 and the cell culture vessel 10 shown in FIG. 2, the outer peripheral edge 4 (outer peripheral wall 13) and the inner peripheral edge 5 (inner peripheral wall 14) that compose the annular culture space are circular. In the cell culture vessel according to the present invention, the outer peripheral edge (outer peripheral wall) and inner peripheral edge (inner peripheral wall) that compose the annular culture space when viewed from overhead are not limited to a circular shape. For example, in the case of using the cell culture vessel for rotary culturing, the outer peripheral edge (outer peripheral wall) and inner peripheral edge (inner peripheral wall) may be in the shape of, for example, a polygon within a range that does not impair rotary culturing.

In this manner, the cell culture vessel of the present invention facilitates the construction of a closed system. Cells cultured with the vessel of the present invention as well as proteins and the like are at little risk to contamination by general bacteria. In addition, cells cultured with the vessel of the present invention as well as proteins and the like are at little risk to viral contamination from the body fluid of another person attributable to cross-contamination.

The cell culture vessel of the present invention can be used for culturing suspended cells in particular. The cell culture vessel of the present invention can be used as a vessel for culturing adherent cells or stimulating suspended cells if the vessel is coated with various types of proteins.

The cell culture vessel of the present invention is preferable for rotary culturing. Applications of the cell culture vessel of the present invention are not limited to rotary culturing. Application of the cell culture vessel of the present invention may be any arbitrary application. Moreover, a cell culture vessel containing medium can be produced by preliminarily sealing a liquid medium and the like in the cell culture vessel of the present invention.

### INDUSTRIAL APPLICABILITY

Since the cell culture vessel of the present invention is provided with a vessel portion having a sealed culture space, the risk of contamination during cell culturing can be reduced. In addition, the cell culture vessel of the present invention is preferable for rotary culturing since it allows the generation of a rotational flow along the annular culture space in liquid medium contained within the vessel.

### BRIEF DESCRIPTION OF THE REFERENCE SYMBOLS

1 Cell culture vessel
2 First vessel wall
3 Second vessel wall
4 Outer peripheral edge
5 Inner peripheral edge
6 Port
7 Tube
8 Outlet port
9 Cap
10 Cell culture vessel
11 First vessel wall
12 Second vessel wall
13 Outer peripheral wall
14 Inner peripheral wall
15 Culture space
16 Port
17 Tube
18 Outlet port
19 Cap
20 Jig
21 Connecting bolts (holding means)
22 Nuts (holding means)
23 Washers (holding means)
24 First plate
25 Second plate

1. A cell culture vessel, comprising:
a vessel portion having an annular sealed culture space when viewed from overhead; and
a port portion connecting the inner portion and outer portion of the culture space; wherein,
the vessel portion is provided with a first vessel wall serving as the upper side during culturing and a second vessel wall serving as the lower side during culturing.

2. The cell culture vessel according to claim 1, wherein the first vessel wall has flexibility.

3. The cell culture vessel according to claim 1, wherein both the first vessel wall and the second vessel wall have flexibility.

4. The cell culture vessel according to claim 1, wherein the first vessel wall has flexibility and the second vessel wall has a shape-retaining property.

5. The cell culture vessel according to claim 4, provided with an annular outer peripheral wall rising from the outer periphery of the second vessel wall and an annular inner peripheral wall rising from the inner periphery of the second vessel wall.

6. The cell culture vessel according to claim 1, wherein both the first vessel wall and the second vessel wall have a shape-retaining property.

7. The cell culture vessel according to claim 2, wherein the inner surface of the first vessel wall and the inner surface of the second vessel wall are substantially adhered to each other when contents containing a gas are discharged from inside the vessel portion.

8. The cell culture vessel according to claim 1, wherein at least one of the first vessel wall and the second vessel wall has oxygen permeability.

9. A jig for fixing the cell culture vessel of claim 1 in position, comprising:
a first plate and a second plate for clamping the vessel portion, and
a holding means for holding the first plate and the second plate in a state of clamping the vessel portion.
